(19) United States
(12) Patent Application Publication
(10) Pub. No.: US 2005/0112157 A1
(43) Pub. Date: May 26, 2005

(54) Foaming cleansing emulsions containing starch

(75) Inventors: Stephan Ruppert, Hamburg (DE); Jorg Kuther, Pinneberg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG.

(21) Appl. No.: 10/963,418

(22) Filed: Oct. 12, 2004

Related U.S. Application Data
(63) Continuation of application No. PCT/EP03/03611, filed on Apr. 8, 2003.

(30) Foreign Application Priority Data
Apr. 11, 2002 (DE) 102 16 510.6

(51) Int. Cl. A61K 7/06; A61K 7/11; A61K 7/075; A61K 7/08

(52) U.S. Cl. 424/401; 424/70.13; 424/70.23; 424/70.24

(57) ABSTRACT

The present invention is a cosmetic cleansing emulsion comprising: a) one or more surfactants with an HLB value of greater than or equal to 16; b) one or more pregelatinized, crosslinked starch derivatives: and c) one or more oil phase components. Optionally, the cosmetic cleansing emulsion further comprises cosmetic active ingredients, auxiliaries and additives. Also encompassed by the invention are hair and body care compositions incorporating such cosmetic cleansing emulsions.

### CROSS-REFERENCE TO RELATED APPLICATIONS

This is a continuation application of PCT/EP03/0361 1, filed Apr. 8, 2003, which is incorporated herein by reference in its entirety, and also claims the benefit of Germany Priority Application No. 102 16 510.6, filed Apr. 11, 2002.

### FIELD OF THE INVENTION

The present invention relates to cosmetic cleansing emulsions comprising
a) one or more surfactants with a HLB value of greater than or equal 16,
b) one or more pregelatinized, crosslinked starch derivatives,
c) one or more oil phase components,
   and, optionally, further cosmetic active ingredients, auxiliaries and additives.

### BACKGROUND OF THE INVENTION

The production of cosmetic cleansing compositions has been showing a rising trend for years. This is to be attributed in particular to the increasing health awareness and hygiene requirement by the consumer.

Cleansing means the removal of (environmental) dirt and thus brings about an increase in psychological and physical wellbeing. The cleansing of the surface of skin and hair is a very complex operation which depends on many parameters. On the one hand, substances originating from the outside, such as, for example, hydrocarbons or inorganic pigments from very diverse surroundings, as well as residues of cosmetics and also undesired microorganisms are to be removed as completely as possible. On the other hand, excretions endogenous to the body, such as perspiration, sebum, skin and hair flakes are to be washed away without far-reaching interventions into the physiological equilibrium.

Cosmetic or dermatological cleansing preparations are so-called “rinse off” preparations which are rinsed off the skin following application. They are generally applied to the areas of the body to be cleansed in the form of a foam with water. The basis of all cosmetic, or dermatological cleansing preparations is washing-active surfactants. Surfactants are amphiphilic substances which are able to dissolve organic, nonpolar substances in water. They are characterized by ambivalent behavior toward water and lipids: the surfactant molecule contains in each case at least one hydrophilic group and one lipophilic group, which permit positioning at the interface between these two classes of substance. In this way, surfactants reduce the surface tension of water, wet the skin, facilitate soil removal and dissolution, provide for easy rinsing and—if desired—also for foam regulation. This is the basis for the soil removal of lipid-containing soilings.

The hydrophilic moieties of a surfactant molecule are mostly polar functional groups, for example —COO⁻, —OSO₃², —SO₃⁻, whilst the hydrophobic moieties are usually nonpolar hydrocarbon radicals. Surfactants are generally classified according to the type and charge of the hydrophilic molecular moiety. In this respect, there are four different groups:
anionic surfactants,
cationic surfactants,
amphoteric surfactants and
nonionic surfactants.

Anionic surfactants generally have carboxylate, sulfate, or sulfonate groups as functional groups. In aqueous solution in an acidic or neutral medium they form negatively charged organic ions. Cationic surfactants are almost exclusively characterized by the presence of a quaternary ammonium group. In aqueous solution in an acidic or neutral medium they form positively charged organic ions. Amphoteric surfactants contain both anionic and cationic groups and, in aqueous solution, accordingly behave as anionic or cationic surfactants, depending on the pH. In a highly acidic medium, they have a positive charge and in an alkaline medium, a negative charge. In the neutral pH range, however, they are zwitterionic, as the following example illustrates:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ B⁺ | (at pH = 12) | B⁺ = any cation, e.g. Na⁺ |

Polyether chains are typical nonionic surfactants. Nonionic surfactants do not form ions in an aqueous medium.

It is understandable that washing-active surfactants which are intended to cleanse the skin and hair of greasy and water-soluble dirt constituents also have a de-fatting action on the normal skin lipids. In any cleansing of the skin, intercorneocytic lipids and sebum constituents are also removed to a varying degree. This means that the natural water/lipid mantle of the skin is more or less destroyed during any washing operation. This can lead, particularly in the case of extreme defatting, to a short-term change in the barrier function of the skin, where, of course, also the particular condition of the area of skin treated is of considerable influence on the changes shown. For example, the skin thickness, the number of sebaceous glands and sweat glands and the sensitivity associated therewith can vary considerably.

In principle, it is accordingly regarded as a requirement of washing-active surfactants that they are as biologically inactive as possible in order to avoid undesired side-effects. They should display their cleansing action with optimum mildness, best skin compatibility, and low de-fatting.

There has hitherto been no lack of attempts to find suitable cleansing preparations which regenerate or “re-fat” the skin at the same time coupled with good cleansing power. However, the performance achieved often remains below that expected, meaning that the user generally has to resort to separate care products which are applied to the skin after cleansing, where they remain (so-called “leave-on” products).

Cosmetic cleansing compositions usually comprise mixtures of surfactants of various types. The choice is orientated primarily to the skin compatibility and the desired cosmetic performance of the surfactants. In addition, foaming power, ability to be formulated, and a favorable performance/cost ratio play an important role.

Liquid soaps or washing lotions are not only used for cleansing the hands, but generally also for the whole body, including the face. They are accordingly also suitable for use as a shower preparation. In the development of these products, the dermatological requirements are foremost, since the skin comes into intensive contact with the concentrated surfactant solution. Particular value is therefore placed on the choice of mild surfactants in low concentration. Further criteria are furthermore a good foaming power, a pleasant, refreshing fragrance and the simultaneous care of the skin. Washing lotions, and in particular shower baths, generally have viscosities of from about 3,000 to 10,000 mPa·s, which on the one hand allow good dispersibility of the product with rapid foaming, but on the other hand should be high enough in order to allow flawless application by hand or flannel.

Liquid soaps or washing lotions are generally characterized by a greater or lesser water content, but as a rule develop no noteworthy care effect since they only have a low oil content.

A relatively new technical development concerns surfactant-containing shower preparations with a high oil content. German laid-open specification 44 24 210 in this connection describes cosmetic or dermatological shower preparations with a surfactant content of at most 55% by weight and an oil content of more than 45% by weight, the preparations being essentially water-free. Owing to the high oil content, these preparations have a regenerating effect with respect to the general condition of the skin. In this case, they at the same time have good foam development and high cleansing power.

In addition, WO 96/17591 describes foaming liquid skin cleansing compositions which comprise the following substances: 5 to 30% by weight of a moisture-donating active ingredient, which has a Vaughan solubility parameter (VSP) of 5 to 10; 0.3 to 5% by weight of a water-dispersible gel-forming polymer; 5 to 30% by weight of a synthetic surface-active substance; 0 to 15% by weight of a C₈ to C₁₄ fatty acid soap; and water; where the preparations have a lipid deposition value (LDV) of at least 5 to 1,000 and in which the synthetic surface-active substance and the soap have a common CMC equilibrium surface tension value of 15 to 50. However, this specification was unable to point the way to the present invention.

For the cleansing and simultaneous care of the skin, the prior art also recognizes emulsion-based cleansing products. These are formulated by stabilizing the emulsion with emulsifiers and subsequently tailoring a surfactant system.

Emulsifiers also have an amphiphilic structure, and are thus comparable to the surfactants as far as the structure is concerned. Emulsifiers allow or facilitate the uniform distribution of two or more mutually immiscible phases and at the same time prevent their separation. Since emulsions are in general destroyed by the addition of surfactants, the choice of surfactant system is severely restricted, and the cleansing preparations obtained are based on expensive and complicated formulations.

Distinguishing washing-active surfactants from emulsifiers can therefore be complicated.

At the end of the 1940s a system was developed which was intended to facilitate the choice of emulsifiers. Each emulsifier is given a so-called HLB value (a dimensionless number between 0 and 20) which indicates whether a preferred water-solubility or oil-solubility is present. Numbers below 9 indicate oil-soluble, hydrophobic emulsifiers, numbers above 11 water-soluble, hydrophilic emulsifiers. The HLB value says something about the equilibrium of the size and strength of the hydrophilic and the lipophilic groups of an emulsifier. It can be derived from these considerations that also the effectiveness of an emulsifier can be characterized by its HLB value. The following list shows the relationship between HLB value and possible field of application:

| HLB value | Field of application |
|---|---|
| 0 to 3 | Antifoam |
| 3 to 8 | W/O emulsifier |
| 7 to 9 | Wetting agent |
| 8 to 18 | O/W emulsifier |
| 12 to 18 | Solubility promoter |

The HLB value of an emulsifier can also be composed of increments, where the HLB increments for the various hydrophilic and hydrophobic groups from which a molecule is composed can be found in tables. In this way, HLB values can in principle also be determined for washing-active surfactants, although the HLB system was originally only conceived for emulsifiers. It is found that washing-active substances generally have HLB values which are significantly greater than 20.

One way of stabilizing surfactant-containing cleansing emulsions against the creaming of the oil drops is to use gel formers which structure the water phase. A central problem in formulating foaming emulsions is the choice of gel former which is compatible both with hydrophilic, in particular ionic, surfactants, and also with oils. Known gel formers generally do not form storage-stable gel networks in the presence of surfactants and oils, meaning that instabilities of the formulations arise. In particular, the gel formation of anionic gel formers is generally very considerably disturbed by anionic surfactants, meaning that extremely high gel former concentrations have to be used.

According to the prior art, to form gel networks in cosmetic cleansing preparations, recourse is made to polyacrylates as gel formers. EP 00112547 describes the stabilization of cleansing emulsions by polyacrylates. With regard to their biological compatibility and, in particular, degradability, and their consumer acceptance, however, it is desirable to replace polyacrylates with other (natural) substances, or to reduce their concentration in the formulations.

### SUMMARY OF THE INVENTION

It was therefore the object of the present invention to provide cleansing preparations based on emulsions which overcome the disadvantages of the prior art and which are accordingly based on simple and cost-effective formulations. The preparations were additionally to have a high care effect without the cleansing effect taking second place.

The object is achieved by cosmetic cleansing emulsion comprising
a) one or more surfactants with an HLB value of greater than or equal to 16,
b) one or more pregelatinized, crosslinked starch derivatives,
c) one or more oil phase,components,
   and, optionally, further cosmetic active ingredients, auxiliaries and additives.

The products according to the invention are characterized by an extremely pleasant smooth and silky feel on the skin during and after application.

### DETAILED DESCRIPTION OF THE PREFERRED EMBODIMENTS

In this connection, it is advantageous according to the invention if the cosmetic cleansing emulsion comprises surfactants in a concentration of from 1 to 20% by weight and very particularly preferably in a concentration of from 5 to 15% by weight, based on the total weight of the preparation.

Although U.S. Pat. No. 6,248,338 describes aqueous surfactant systems containing pregelatinized, crosslinked starch derivatives, this specification was unable to point the way to the present invention.

It is advantageous according to the invention if one or more pregelatinized, crosslinked starch derivatives are present in the cleansing emulsion according to the invention in a concentration from 0.1 to 20% by weight, preferably in a concentration of from 0.3 to 15% by weight and very particularly preferably in a concentration of from 0.5 to 10% by weight, based on the total weight of the preparation.

It is also advantageous according to the invention if the oil phase is present in the cleansing emulsion in a concentration of from 5 to 80% by weight, particularly preferably in a concentration of from 30 to 75% by weight, and very particularly preferably in a concentration of from 40 to 50% by weight.

It is advantageous according to the invention if the pregelatinized, crosslinked starch derivatives used are hydroxypropylated phosphate esters. Particularly advantageous starch derivatives are those as described in U.S. Pat. No. 6,248,338, particularly advantageously hydroxypropyl distarch phosphate. Very particular preference is given here to the use of a hydroxypropyl distarch phosphate (CAS number 113894-92-1), as is sold as the product Structure® XL from National Starch.

Advantageous washing-active anionic surfactants for the purposes of the present invention are:
acylamino acids and salts thereof, such as
   acylglutamates, in particular sodium acylglutamate
   sarcosinates, for example myristoyl sarcosine, TEA lauroyl sarcosinate, sodium lauroyl sarcosinate and sodium cocoyl sarcosinate,
sulfonic acids and salts thereof, such as
   acyl isethionates, e.g. sodium/ammonium cocoyl isethionate,
   sulfosuccinates, for example dioctyl sodium sulfosuccinate, disodium laureth sulfosuccinate, disodium lauryl sulfosuccinate and disodium undecylenamido MEA sulfosuccinate,
and sulfuric esters, such as
   alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Advantageous washing-active cationic surfactants for the purposes of the present invention are quaternary surfactants. Quaternary surfactants contain at least one N atom which is covalently bonded to four alkyl or aryl groups. Benzalkonium chloride, alkylbetaine, alkylamidopropylbetaine and alkylamidopropylhydroxysultaine are advantageous.

Advantageous washing-active amphoteric surfactants for the purposes of the present invention are:
acyl-/dialkylethylenediamines, for example sodium acyl amphoacetate, disodium acyl amphodipropionate, disodium alkylamphodiacetate, sodium acyl amphohydroxypropylsulfonate, disodium acyl amphodiacetate and sodium acyl amphopropionate.

Advantageous washing-active nonionic surfactants for the purposes of the present invention are:
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers and alkyl polyglycosides, such as lauryl glycoside, decyl glycoside and cocoglycoside.

Further advantageous anionic surfactants are:
taurates, for example sodium lauroyl taurate and sodium methyl cocoyl taurate,
ether carboxylic acids, for example sodium laureth-13 carboxylate and sodium PEG-6 cocamide carboxylate,
phosphoric esters and salts, such as, for example, DEA-oleth-10 phosphate and dilaureth-4 phosphate,
alkylsulfonates, for example sodium cocosmonoglyceride sulfate, sodium C₁₂₋₁₄-olefinsulfonate, sodium lauryl sulfoacetate and magnesium PEG-3 cocamidesulfate.

Further advantageous amphoteric surfactants are:
N-alkylamino acids, for example aminopropylalkylglutamide, alkylaminopropionic acid, sodium alkylimidodipropionate and lauroamphocarboxyglycinate.

Further advantageous nonionic surfactants are alcohols.

Further suitable anionic surfactants for the purposes of the present invention are also:
acyl glutamates, such as di-TEA-palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen,
   and carboxylic acids and derivatives, such as
for example lauric acid, aluminum stearate, magnesium alkanolate and zinc undecylenate,
ester carboxylic acids, for example calcium stearoyl lactylate, laureth-6 citrate and sodium PEG4 lauramide carboxylate,
alkylarylsulfonates.

Further suitable cationic surfactants for the purposes of the present invention are also:
alkylamines,
alkylimidazoles and
ethoxylated amines.

Further suitable nonionic surfactants for the purposes of the present invention are also amine oxides, such as cocoamidopropylamine oxide.

It is particularly preferred according to the invention if the surfactants used are anionic surfactants. Here, the use of alkyl ether sulfates, in particular sodium lauryl ether sulfate or myristyl ether sulfate, is very particularly preferred according to the invention.

One embodiment of the present invention which is particularly advantageous according to the present invention is, in particular, the combination of the surfactants of sodium lauryl ether sulfate with sodium cocoyl glutamate and/or decyl glycoside.

According to the invention, polysorbates can also advantageously be incorporated into the emulsion as washing-active agents.

Besides water, according to the invention, the preparation according to the invention can also comprise other ingredients as aqueous phase of the emulsion, for example alcohols, diols or polyols with low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol and glycerol.

The oil phase of the cosmetic or dermatological cleansing emulsions for the purposes of the present invention is advantageously chosen from the group of esters of saturated and/or unsaturated, branched and/or unbranched alkane carboxylic acids with a chain length of from 3 to 30 carbon atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate, and synthetic, semisynthetic, and natural mixtures of such esters, e.g. jojoba oil.

In addition, the oil phase can advantageously be chosen from the group of branched and unbranched hydrocarbons and hydrocarbon waxes, the silicone oils, the dialkyl ethers, the group of saturated or unsaturated, branched or unbranched alcohols, and the fatty acid triglycerides, namely the triglycerol esters of saturated and/or unsaturated, branched and/or unbranched alkane carboxylic acids with a chain length of from 8 to 24, in particular 12 to 18 carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any desired mixtures of such oil and wax components are also to be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyidodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

Advantageously, the oil phase can also have a content of cyclic or linear silicone oils or consist entirely of such oils, where, however, it is preferred to use an additional content of other oil phase components apart from the silicone oil or the silicone oils.

Advantageously, cyclomethicone (octamethylcyclotetrasiloxane) is used as the silicone oil to be used according to the invention. However, other silicone oils are also to be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

The oil phase is also advantageously chosen from the group of phospholipids. The phospholipids are phosphoric esters of acylated glycerols. Of very great importance among the phosphatidyl cholines are, for example, the lecithins, which are characterized by the general structure 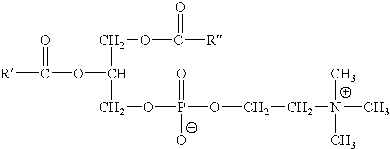
where R′ and R″ are typically unbranched aliphatic radicals having 15 or 17 carbon atoms and up to 4 cis double bonds.

It is advantageous according to the invention if the cosmetic cleansing emulsion comprises one or more polyacrylates.

Advantageous polyacrylates according to the invention are polymers of acrylic acid, in particular those which are chosen from the group of so-called carbomers or carbopols (Carbopol® is actually a registered trademark of the B.F. Goodrich company). Polyacrylates are compounds of the general structural formula 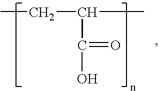
whose molecular weight can be between about 400,000 and more than 4,000,000. The group of polyacrylates also includes acrylate/alkyl acrylate copolymers, for example those which are characterized by the following structure: 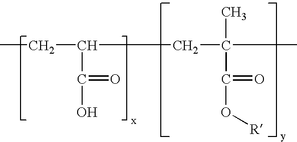
In this R′ is a long-chain alkyl radical and x and y are numbers which symbolize the particular stoichiometric proportion of the respective comonomers. These polyacrylates are also advantageous for the purposes of the present invention.

Advantageous carbopols are, for example, the grades 907, 910, 934, 940, 941, 951, 954, 980, 981, 1342, 1382, 2984 and 5984 or alternatively the grades ETD (Easy-to-disperse) 2001, 2020, 2050, Aqua-SF1 where these compounds can be present individually or in any combinations with one another.

Also advantageous for the purposes of the present invention are the copolymers of C₁₀₋₃₀-alkyl acrylates and one or more monomers of acrylic acid, methacrylic acid or esters thereof comparable to the acrylate/alkyl acrylate copolymers. The INCI name of such compounds is “Acrylates/C 10-30 Alkyl Acrylate Crosspolymer”. Those obtainable under the trade names Pemulen TR1 and Pemulen TR2 from the B.F. Goodrich company are particularly advantageous.

According to the invention, it is particularly advantageous if the polyacrylates used are C₁₀ to C₃₀-alkyl acrylate copolymers.

It is advantageous for the purposes of the present invention when the content of one or more polyacrylates in the cosmetic or dermatological cleansing emulsion is chosen from the range from 0.5 to 2% by weight, very particularly advantageously from 0.7 to 1.5% by weight, in each case based on the total weight of the preparations.

Apart from the abovementioned substances, the compositions optionally comprise, in accordance with the invention, the additives customary in cosmetics, for example perfume, dyes, antimicrobial substances, re-fatting agents, complexing and sequestering agents, pearlizing agents, plant extracts, vitamins, active ingredients, preservatives, bactericides, pigments which have a coloring action, thickeners, softening, moisturizing and/or humectant substances, or other customary constituents of a cosmetic or dermatological formulation, such as alcohols, polyols, polymers, foam stabilizers, electrolytes, organic solvents or silicone derivatives.

An additional content of antioxidants is generally preferred. According to the invention, favorable antioxidants which can be used are all antioxidants which are suitable or customary for cosmetic and/or dermatological applications.

The active ingredients, auxiliaries and additives which can be used advantageously according to the invention are in no way limited to the substances and compounds mentioned by name.

It is also advantageous according to the invention if effect substances (e.g. colored beads and/or active ingredient beads, glitter substances, etc.) are added to the preparations according to the invention and/or the preparation is provided with air bubbles and blisters which are stable in the emulsion.

The use of the cosmetic cleansing emulsion according to the invention as foam bath, shower bath (shower gel), tub bath and/or hair-washing composition (shampoo) is in accordance with the invention.

The use of the cosmetic cleansing emulsion for the prophylaxis and/or treatment of inflammatory skin conditions and/or for skin protection in the case of sensitively determined and dry skin (i.e. as dermatological preparation) is also in accordance with the invention.

The examples below, in which washing preparations for hair care and body care are described, are intended to illustrate the compositions according to the invention, but without any restriction of the invention to these examples being intended. The numerical values in the examples are percentages by weight, based on the total weight of the particular preparations.

### FORMULATION EXAMPLES 1-5

| | 1 | 2 | 3 | 4 | 5 |
|---|---|---|---|---|---|
| Paraffin oil | 46% | 14% | 20% | 20% | 25% |
| Soybean oil | 24.3% | 36% | 20% | 20% | 25% |
| Sodium lauryl ether sulfate | 7.35% | 12.3% | 11% | 11% | 11% |
| Sodium cocoyl glutamate | — | 2.5% | 1% | — | — |
| Decyl glucoside | — | — | — | — | 2% |
| Sodium cocoamphoacetate | — | — | 3% | — | — |
| Hydroxypropyl starch | 1% | 2% | 5% | 1% | 7% |
| phosphate ester | | | | | |
| (Structure XL) | | | | | |
| Sodium benzoate | 0.3% | 0.3% | 0.3% | — | 0.3% |
| Sodium salicylate | 0.2% | 0.2% | 0.2% | — | 0.2% |
| Acrylates/C10-C30 alkyl | — | — | 1% | 1% | 0.8% |
| acrylate crosspolymer | | | | | |
| Sodium hydroxide | — | — | 0.2% | 0.2% | 0.2% |
| Phenoxyethanol | — | — | — | 0.5% | — |
| Parabens | — | — | — | 0.2% | — |
| Perfume | q.s. | q.s. | q.s. | q.s. | q.s. |
| Water | ad | ad | ad | ad | ad |
| | 100 | 100 | 100 | 100 | 100 |

### FORMULATION EXAMPLES 6-10

| | 6 | 7 | 8 | 9 | 10 |
|---|---|---|---|---|---|
| Paraffin Oil | — | 10% | 65% | 3% | 5% |
| Soybean oil | 10% | — | 5% | 1% | 5% |
| Sodium lauryl ether sulfate | 7.35% | 12.3% | — | — | 2% |
| Sodium cocoyl glutamate | 2% | — | 7% | — | 2% |
| Decyl glucoside | — | — | — | 5% | — |
| Cocamidopropylbetaine | 2.5% | 3% | — | — | — |
| Hydroxypropyl starch | 2% | 8% | 10% | 0.8% | 1% |
| phosphate | | | | | |
| ester (Structure XL) | | | | | |
| Sodium benzoate | 0.3% | 0.3% | 0.3% | — | 0.3% |
| Sodium salicylate | 0.2% | 0.2% | 0.2% | — | 0.2% |
| Acrylates/C10-C30 alkyl | — | — | 0.7% | 0.4% | 0.3% |
| acrylate crosspolymer | | | | | |
| Sodium hydroxide | — | — | 0.2% | 0.2% | 0.2% |
| Phenoxyethanol | — | — | — | 0.5% | — |
| Parabens | — | — | — | 0.2% | — |
| Perfume | q.s. | q.s. | q.s. | q.s. | q.s. |
| Water | ad | ad | ad | ad | ad |
| | 100 | 100 | 100 | 100 | 100 |

1. A cosmetic cleansing emulsion comprising:
a) one or more surfactants having an HLB value of greater than or equal to 16,
b) one or more pregelatinized, crosslinked starch derivatives, and
c) one or more oil phase components.

2. The cosmetic cleansing emulsion of claim 1, wherein the one or more surfactants are present in a concentration of from 1 to 20% by weight based on the total weight of the emulsion.

3. The cosmetic cleansing emulsion of claim 1, wherein the one or more pregelatinized, crosslinked starch derivatives are present in a concentration of from 0.1 to 20% by weight based on the total weight of the emulsion.

4. The cosmetic cleansing emulsion of claim 1, wherein the one or more oil phase components are present in a concentration of from 5 to 80% by weight based on the total weight of the emulsion.

5. The cosmetic cleansing emulsion of claim 1, wherein the one or more pregelatinized, crosslinked starch derivatives include one or more hydroxypropylated phosphate esters.

6. The cosmetic cleansing emulsion of claim 5, wherein the one or more pregelatinized, crosslinked starch derivatives include hydroxypropyl distarch phosphate.

7. The cosmetic cleansing emulsion of claim 1, wherein the one or more surfactants include one or more anionic surfactants.

8. The cosmetic cleansing emulsion of claim 7, wherein the one or more anionic surfactants include one or more alkyl ether sulfates.

9. The cosmetic cleansing emulsion of claim 8, wherein the one or more alkyl ether sulfates include sodium lauryl ether sulfate.

10. The cosmetic cleansing emulsion of claim 8, wherein the one or more alkyl ether sulfates include sodium myristyl ether sulfate.

11. The cosmetic cleansing emulsion of claim 1, further comprising one or more polyacrylates.

12. The cosmetic cleansing emulsion of claim 11, wherein the one or more polyacrylates include one or more C₁₀ to C₃₀-alkyl acrylate copolymers.

13. The cosmetic cleansing emulsion of claim 11, wherein the one or more polyacrylates are present in a concentration of 0.5 to 2% by weight based on the total weight of the emulsion.

14. A composition comprising a cosmetic cleansing emulsion of claim 1, wherein the composition is selected from the group consisting of a shower bath composition, a tub bath composition, and a hair-washing shampoo composition.
